(19) United States
(12) Patent Application Publication
(10) Pub. No.: US 2007/0243240 A9
(43) Pub. Date: Oct. 18, 2007

(54) Transdermal therapeutic system

(76) Inventors: Fred Windt-Hanke, Berlin (DE); Clemens Gunther, Berlin (DE); Reinhard Horowski, Berlin (DE); Johannes Tack, Berlin (DE); Adalbert Engfer, Berlin (DE); Katalin Tisa Bostedt, Pullach (DE); Dirk Schenk, Dietramszell (DE)

Correspondence Address: Michelle S. Marks;SHAW PITTMAN LLP, 1650 Tysons Boulevard, McLean, VA, 22102, US

(21) Appl. No.: 11/087,754

(22) Filed: Mar. 24, 2005

Related U.S. Application Data
(63) Continuation-in-part of application No. PCT/DE04/01133, filed on May 30, 2004.
(63) Continuation-in-part of application No. 10/362,248, filed on Jul. 7, 2003.
(63) Continuation-in-part of application No. 10/362,183, filed on Jul. 21, 2003.
(63) Continuation-in-part of application No. 10/362,182, filed on Jul. 3, 2003, now Pat. No. 7,258,871.

(30) Foreign Application Priority Data
Oct. 20, 2000 (DE) 100 53 397.3
Sep. 3, 2003 (DE) 10341317
Aug. 24, 2000 (DE) 10043321.9

(51) Int. Cl. A61K 9/70

(52) U.S. Cl. 424/449

(57) ABSTRACT

The invention concerns a transdermal therapeutic system containing ergoline derivatives, preferably lisuride, with a stabilized ergoline compound. Stabilization of the oxidation sensitive ergoline combination is done through a combination of at least one fat-soluble, radical-trapping antioxidant, preferably Di-tert.-butylmethylphenols, Di-tert.-butylmetoxyphenols, tocopherols or ubichinones and a basic polymer. Use of a transdermal therapeutic system (TTS) comprising a pharmaceutical layer containing at least one matrix having an active ingredient and/or an active ingredient reservoir; a diffusion barrier that is permeable to said active ingredient and arranged on the skin side of the active ingredient reservoir; and an ergoline derivative or salt thereof as an active ingredient for producing an agent for obtaining and maintaining the circadian rhythm under dopamine therapy. The invention relates to the use of a transdermal therapeutic system (TTS) comprising a medicinal layer, which contains at least one matrix comprising an active ingredient and/or an active ingredient reservoir and a diffusion barrier situated on the skin side of the active ingredient reservoir and permeable to active ingredients, in addition to, an ergoline-derivative or physiologically compatible salt with an acid thereof, as an active ingredient, for producing a means for treating the Restless Leg Syndrome and Periodic Limb Movement Disorder. The invention relates to the use of a dopamine agonist in the form of an agent consisting of at least two spatially discrete compositions, of which one is a transdermal therapeutic system (TTS) containing the dopaminergic agent and another one or more are preparations for oral and/or parenteral application containing that same dopaminergic agent for the treatment of dopaminergically treatable diseases with the following elements: a) the TTS is continuously applied, b) within the duration of application in a) the composition for oral or parenteral dosage is administered.

This application claims priority under 35 U.S.C. § 120 to U.S. Ser. No. 10/362,248 filed Jul. 7, 2003; PCT/EP01/09824 filed Aug. 24, 2001; Ser. No. 10/362,183 filed Jul. 21, 2003; PCT/EP01/09823 filed Aug. 24, 2001; U.S. Ser. No. 10/362,182 filed Jul. 3, 2003, PCT/EP01/09826 filed Aug. 24, 2001, and PCT/DE2004/001133 filed Jun. 7, 2004, each of which is incorporated herein by reference in its entirety.

### BACKGROUND

### Field of the Invention

The present invention relates generally to a transdermal therapeutic device and system for delivering an ergoline compound. More specifically, the present invention relates to an extended shelf-life transdermal therapeutic device for delivering an ergoline compound transdermally, over a period of days.

### DETAILED DESCRIPTION OF THE INVENTION

Ergoline Transdermal Therapeutic System With Extended Shelf Life

### Description

The invention relates to a medication for transdermal application consisting of an impermeable backing layer, a matrix containing an ergoline compound and possibly a penetration enhancer, possibly a diffusion barrier covering the matrix, a layer of adhesive permeable for these substances and a peel-off protective cover. The ergoline derivatives, preferably lisuride, in transdermal therapeutic systems need to be stabilized.

Transdermal therapeutic systems containing ergoline derivatives have been used to treat diseases caused by disorders of the dopaminergic system (WO 92/20339, WO 91/00746). They appear to be especially suited for the treatment of Parkinson's disease, Parkinsonism, Restless Legs Syndrome, as prophylaxis for Premenstrual Syndrome and as a lactation inhibitor (DE 100 43 321). Sometimes they are also intended for migraine prophylaxis, where a well-tolerated, constant therapy is desired.

Transdermal therapeutic systems can, for instance, be structured as so-called matrix systems. Matrix systems typically consist of an impermeable backing layer, the matrix with the drug formulation imbedded or dissolved and, if the matrix does not stick to skin on its own, a layer of adhesive and a peel-off protective cover.

To reach a defined, continuous flow, the drug is usually combined with suitable excipients, such as solvents, penetration enhancers and crystallization inhibitors.

Transdermal therapeutic systems with oxidation sensitive drugs are not very stable. Improvement of the stability of these systems is described in DE 100 54 713 A1. In this description all of the system's formulation components are selected in a way that the total of their peroxide numbers (as an indicator of its oxidizability) is not more than 20. This means, however, that the contents that can be considered, are limited or that it would require elaborate and costly preparatory treatments of the individual excipients with sodium hydrogen sulfite solutions to destroy the existing peroxides.

But the problem with preparations containing ergoline derivatives up to now has been the instability of the drug itself. Transdermal therapeutic systems containing ergoline derivatives after some time show discolorings, typically correlated with a decay of the active drug content. This is caused by the rather high oxidation sensitivity of ergoline derivatives. So lisuride, for instance, is being oxidized even without light at the nitrogen in position 6 of the ring system.

This leads to skin irritation, especially in the case of long-term application. Controlled dosing is also not possible anymore due to the unknown reduction of the drug content.

The antioxidants commonly used for stabilizing, such as citric acid, ascorbic acid, sodium sulfite, alkyl gallate, ascorbyl palmitate and others, do not result in any improvement.

The aim of the present invention is the creation of a transdermal therapeutic system containing an ergoline derivative, which is stable on storage and does not allow oxidative degradation of the drug and which can thus remain on the skin without irritations even over long periods of time.

According to this invention the task is solved by stabilizing the ergoline derivatives in a transdermal therapeutic system through combining at least one fat-soluble, radical-trapping antioxidant, preferably Di-tert.-butylmethylphenols, Di-tert.-butylmetoxyphenols, tocopherols or ubichinones and a basic polymer.

Investigations have shown that the presence of one of the above mentioned antioxidants alone does not result in a significant improvement of the stability of the ergoline derivatives.

Transdermal therapeutic systems, in which there is also a basic polymer present, such as butylmethacrylate-(2-dimethyl aminoethyl)methacrylate-methyl methacrylate-copolymer (Eudragit E 100 by Röhm, Germany), besides the above mentioned antioxidants, display a surprisingly high stability. In this, the basic polymer can be present also in a mixture with the usual other polymers, such as neutral polyacrylates. Moreover, the polymer mixture can contain common adhesiveness enhancers (i.e., resins or polyacrylates) to improve the adhesive strength.

The systems according to this invention usually have an area weight of 2 to 10 mg/cm². This is the sum of all components after drying. The total content of matrix forming polymers is 50% to 95% w/w, preferably 60% to 85%. The portion of other polymers is 5% to 30% w/w, preferably 10% to 20%. The content of antioxidants is between 0.25% and 5% w/w, preferably 0.5% to 1.5%. The portion of the drug is 1% to 10% w/w, preferably 3% to 6%.

The combinations according to this invention have an unexpected synergy effect inhibiting oxidation of ergoline derivatives in transdermal systems.

The stability of the transdermal therapeutic system comprising an ergoline compound is achieved by the presence of at least one fat-soluble or lipophilic antioxidant and a basic polymer in the matrix containing the ergoline compound. Preferably, the ergoline compound is lisuride, the lipophilic antioxidant is selected from the group consisting of Di-tert.-butylmethylphenols, Di-tert.-butylmetoxyphenols, tocopherols and ubichinones and the basic polymer is a basic copolymer such as Eudragit.

Examples of lipophilic antioxidants include, but are not limited to the following: 2,2,5,7,8-pentamethyl-6-chromanol; 2,2′-Azobis (2-amidino-propane) dihydrochloride (AAPH); 2,6-di-tert-butyl-4-methylphenol (BHT); ethanolic BHT; 2-tert-butyl-4-methylphenol; 4-Difluoro-5-(4-phenyl-1,3-butadienyl)-4-bora-3a,4a-diaza-s-indacene-3-undecanoic acid; Á-oryzanol; ascorbyl palmitate; α-carotene; β-carotene; Coenzyme Q10; Coenzyme Q10H2; coenzyme Q9 (CoQ9H2); copper; β-Cryptoxanthin; α-lipoic acid; Lutein/zeaxanthin; luteine; lycopene; malondialdehyde (MDA); meth-6-hydroxy-2,5,7,8-tetramethyl-2-carboxylic acid (Trolox); Nutriene (Tocotrienols); γ-Oryzanol; Retinol; R-tocopherol acetate; R-tocopherol; selenium; α-tocopherol; α-tocopherol acetate; γ-tocopherol; δ-tocopherol; Trolox; ubiquinol; Ubiquinol 10; vitamin E; and zinc.

The following molecules are examples of hydrophilic antioxidants: citric acid, ascorbic acid (vitamin C), sodium sulfite, alkyl gallate, ascorbyl palmitate, Cysteine, methionine, glutathione, Sodium hydrogensulfite), uric acid, TBA-RS, and protein carbonyls.

The following are examples of basic polymers and basic polyacrylates: hydrophilic polyacrylate with basic substituents, GELVA, and Eudragit E100 (dimethyl aminomethyl methacrylate w/neutral methacrylate esters).

### EXAMPLE A

### Stability Testing

Stability investigations are carried out with samples containing combinations of different antioxidants and polymers.

The ergoline compound tested as the drug is lisuride. In addition to lisuride, the samples contained additional ingredients for use in transdermal therapeutic systems.

Preparations of the Samples:

150 g polyvidone and 300 g dibutyl sebacate as softeners and 20 g Foral E105 (hydrated colophonium pentaerthrite ester by Hercules) as tackifier are one after the other stirred into 900 g of a 50% aqueous solution of polymer adhesive in a mixture of 2-propanol and acetone at room temperature. Then 50 g lisuride and 15 g antioxidant are pre-suspended in part of the solvent and added to the adhesive mixture, being stirred constantly. Once it is completely dissolved, the solution is replenished with acetone to achieve the final weight and left sitting for about 24 hours to remove gas bubbles. Afterwards the solution is applied to a siliconized polyester film (Liner Film) with a suitable coating device (i.e., Knife over Roll), so after removing the volatile solvents at 40 to 90° C. an even film with an area weight of about 5 mg/cm² develops. Then it is concealed with a polyester cover foil. The laminate thus achieved is cut into single patches with sizes of 10 cm² each with a suitable stamping device and inserted into light proof pouches of aluminum-paper compound material.

TABLE 1
The composition of the samples tested in % w/w.

sample no.

| | #80 | #81 | #82 | #83 | #84 | #85 | #86 | #87 | #88 | #90 | #98 |
|---|---|---|---|---|---|---|---|---|---|---|---|
| lisuride | 3.3 | 3.2 | 4.0 | 4.0 | 4.0 | 4.0 | 4.0 | 4.0 | 4.0 | 3.0 | 3.0 |
| MA24A (1) | 44.9 | 45.1 | — | — | — | — | — | — | — | 53.0 | 52.0 |
| Eudragit E 100 (2) | — | — | — | — | 85.0 | 85.0 | 68.0 | 68.0 | 60.0 | — | — |
| Durotac DT 387-2510 (3) | — | — | 77.0 | 77.0 | — | — | 17.0 | 17.0 | 15.0 | — | — |
| Ascorbyl palmitate | — | — | — | — | — | — | — | — | — | — | 2.0 |
| Pocopherol | 1.0 | — | 1.0 | — | 1.0 | — | 1.0 | — | 1.0 | 1.0 | — |
| BHT (4) | — | 0.9 | — | 1.0 | — | 1.0 | — | 1.0 | — | — | — |
| Polyvidon | 9.2 | 9.3 | 10.0 | 10.0 | 10.0 | 10.0 | 10.0 | 10.0 | 20.0 | 10.0 | 10.0 |
| Transcutol (5) | 27.0 | 27.0 | — | — | — | — | — | — | — | 25.0 | 25.0 |
| Eutanol (6) | 8.7 | 8.6 | 5.0 | 5.0 | — | — | — | — | — | 8.0 | 8.0 |
| Dimethylacetamide | 5.9 | 5.9 | — | — | — | — | — | — | — | — | — |

(1) Polyisobutylene [Adhesive Research, Ireland]
(2) Butyl methacrylate-(2-diaminoethyl)methacrylate-methacrylate-copolymer (1:2:1) [Röhm, Germany]
(3) neutral polyacrylate [National Starch, USA]
(4) Butylhydroxytoluene (2,6-di-tert.-butyl-4-methylphenol)
(5) Diethylene glycol monoethylether [Gattefossé, France]
(6) 2-hexyldecanol [Cognis, Germany]

Storage: The samples are stored under the following conditions:
a) at 4° C.
b) at 25° C. and 60% humidity
c) at 40° C. and 75% humidity

After one month of storage the concentration of the aminoxide achieved is determined through oxidation at the nitrogen in position 6 of the ergoline ring system (lisuride-N-oxide).

Determination of the aminoxide content: The amount of aminoxide is determined with a HPLC method, showing the following parameters:

Column: Luna C18(11), 100 mm×4.6 mm ID

Pre-column: Phenomenex C18, 4 mm×3 mm ID

Column temperature: 35° C.

Running time: 30 min

Flow rate: 1.20 ml/min

Mobile phase: A: 10 mM TRIS-Buffer, pH 8.7
B: Acetonitrile

Gradient profile: 0 to 25^{th} minute: 12% B
25^{th} to 27^{th} minute: 42% B
28^{th} to 38^{th} minute: 12% B

Detection: Fluorescence Detector

Preparation of the Samples:

One lisuride patch, produced as described in Example A, is shaken in 50 ml solvent (2-propanol) for 15 minutes after weighing and removing the liner film. Then 5 ml of the solution are diluted with a diluent (acetonitril) to the volume of 20 ml. About 2 ml of this solution are centrifugated at 5000 rpm for 2 minutes and the clear resulting solution is being transferred to a HPLC sample vial.

TABLE 2
Creation of aminoxide from lisuride after one month storage

| | Content of lisuride-N-oxide | | |
|---|---|---|---|
| Sample | a) 4° C. | b) 25° C. | c) 40° C. |
| #80: | 0.51 | 1.59 | 2.80 |
| MA24A/Tocopherol | | | |
| #81: | 0.45 | 1.26 | 1.86 |
| MA24A/BHT | | | |
| #82: | 0.70 | 1.21 | 1.49 |
| Durotac/Tocopherol | | | |
| #83: | 0.71 | 1.08 | 1.44 |
| Durotac/BHT | | | |
| #84: | 0 | 0.11 | 0.26 |
| Eudragit/Tocopherol | | | |
| #85: | 0.06 | 0.09 | 0.22 |
| Eudragit/BHT | | | |
| #86: | 0 | 0.14 | 0.37 |
| Eudragit/Durotac/Tocopherol | | | |
| #87: | 0 | 0.14 | — |
| Eudragit/Durotac/BHT | | | |
| #88: | 0.11 | 0.21 | 0.44 |
| Eudragit/Durotac/Tocopherol | | | |
| #90: | — | — | 1.93 |
| MA24A/Tocopherol | | | |
| #98 | 0.27 | 0.58 | — |
| MA24A/Ascorbyl Palmitate | | | |

### EXAMPLE B

Stability investigations are carried out with samples containing combinations of different antioxidants with basic polyacrylates. In this process lisuride is employed as the drug. In addition to this, the samples contain more ingredients usually used in transdermal therapeutic systems.

Preparation of the Samples:

175 g polyvidone and 310 g dibutyl sebacate as softeners and 175 g dodecanol as a co-solvent were consecutively stirred into 1800 g of an about 45% aqueous solution of basic polyacrylate adhesive in acetone at room temperature. Then 80 g lisuride and 17 g antioxidants are pre-suspended in part of the solution and added to the adhesive solution. Once it is completely dissolved, 35 g Foral are added as a tackifier. The solution is replenished with acetone to reach the final weight and is then left sitting for about 24 hours to remove the gas bubbles. After that, the solution is applied to a siliconized polyester sheet (Liner Film) with a suitable coating device (i.e. Knife over Roll), so after taking away the volatile solvents at 40 to 90° C. an even film with an area weight of about 5 mg/cm² develops. Then it is concealed with a polyester cover foil. The laminate thus created is cut into single patches with sizes of 10 cm² each with a suitable stamping device and put into light proof pouches of aluminim-paper compound material.

TABLE 3
Composition of the samples in % w/w

| | Sample #. | | | | |
|---|---|---|---|---|---|
| | #C005 | #151 | #156 | #C001 | #152 |
| Lisuride | 5.0 | 5.0 | 5.0 | 4.0 | 5.0 |
| Polyvidon | 10.0 | 10.0 | 10.0 | 10.0 | 10.0 |
| lauryl alcohol | 15.0 | 15.0 | 15.0 | — | 15.0 |
| Foral 105 E (1) | 2.0 | 2.0 | 2.0 | — | 2.0 |
| BHT (2) | — | — | 1.0 | 0.4 | — |
| Sodium sulfite | — | — | — | — | 0.1 |
| Eudragit E 100 (3) | 68.0 | 68.0 | 67.0 | 85.6 | 67.9 |

(1) hydrated colophonium penta erythritester by Hercules
(2) Butyl hydroxy toluene
(3) Butyl methacrylate-(2-diaminoethyl)methacrylate-methacrylate-copolymer (1:2:1) [Röhm, Germany]

Storage: The samples are stored at 25° C. and 60% humidity and at 40° C. and 75% humidity. After one month's and after three months of storage concentration of the aminoxide is determined.

Determination of the aminoxide content: The aminoxide content of the samples is determined with the HPLC method described in Example A.

The results of the stability tests are presented in Table 4.

TABLE 4
Creation of aminoxide from lisuride after a one month and a three
month storage.

| | content of lisuride-N-oxide in % w/w | | | |
|---|---|---|---|---|
| | 25° C. | 25° C. | 40° C. | 40° C. |
| Sample | 1 month | 3 months | 1 month | 3 months |
| #C005: Eudragit | 0.20 | 0.50 | 1.18 | 3.51 |
| #151: Eudragit | 0.21 | 0.40 | 0.91 | 2.43 |
| #153: Eudragit | 0.21 | 0.48 | 0.92 | 2.00 |
| #156: | 0.14 | — | 0.27 | — |
| Eudragit/BHT | | | | |
| #C001: | 0.10 | 0.14 | 0.38 | 0.44 |
| Eudragit/BHT | | | | |
| #152: | 0.18 | 0.36 | 0.82 | 2.23 |
| Eudragit/Sodium | | | | |
| Sulfite | | | | |

Preferably, the transdermal therapeutic device and system of the present invention is useful as a medicament in the following conditions: migraine prophylaxis, menstrual associated migraine, and as an abortive treatment of migraine to alleviate the symptoms and shorten the duration of migraine, PMS symptoms, hyperprolactinemia, gynecomastia, depression, attention deficit disorder (ADD), smoking cessation, eating disorders and the loss of libido.

Additional therapeutic uses of particular embodiments of the transdermal therapeutic device and system of the present invention include: (i) to suppress bowel movements and diarrhea in carcinoid syndrome and/or irritable bowel syndrome; (ii) to prevent and or treat fibrotic cardiac valvulopathy, which can be drug induced (e.g., by pergolide) and is also the main cause of death in carcinoid patients, and pulmonary hypertension; (iii) prophylaxis of migraine attacks (iv) treatment of migraine; (v) prevention and/or control of the symptoms of premenstrual syndrome (PMS), including but not limited to premenstrual migraine, headaches, breast tension or mastopathy, and edema. The TTS of the present invention containing a lower dose of the ergoline compound is particularly well suited for the therapeutic uses identified in (i)-(v).

One preferred method for preventing and/or treating the symptoms associated with PMS is the application of one transdermal therapeutic device of the present invention per menstrual cycle. The timing of such application is prior to the start of menstruation, such as at about day 24 of the menstrual cycle. This timing can vary per individual and the individual and/or the consulting physician are capable of determining the day in the individual's menstrual cycle for applying the device in order to achieve optimal prevention and/or alleviation of PMS symptoms. In another embodiment of the method of the invention, the device of the invention may also be used in combination with other prolactin-lowering compounds and/or analgesics.

The combination of an oral dopamine agonist (lisuride) and levodopa (approved in some European countries as Restex® for RLS). As higher doses of levodopa (which otherwise is a good treatment which patients like) are linked to longterm side effects (so-called augmentation, i.e. more or longer-lasting involuntary movements when at rest), a strategy of keeping levodopa low (or even only as short-acting rescue medication on top of chronic lisuride patch use) as basic therapy becomes very interesting (analogous to Parkinson's disease, where these combinations (DA agonists and levodopa) now are the preferred treatment). Many patients on chronic dialysis for kidney disease suffer from severe RLS (and/or hormonal disturbances including mild hyperprolactinemia) but cannot tolerate oral dopamine agonists. Such dialysis patients may be treated with low dose lisuride release from a device of the present invention.

The TTS of the present invention may also be used as for the purpose of stabilize or slow progressive neurodegeneration, particularly in Parkinson's disease. The TTS of the present invention containing lisuride may also be used to treat conditions that benefit from continuous administration of a 5-HT1A agonist, a dopamine agonist, a platelet aggregation inhibitor, an anti-oxidant, a radical scavenger, and a glutamate antagonist.

Further Inventions

This invention relates to a transdermal therapeutic system (TTS) comprising a pharmaceutical layer containing at least one matrix having an active ingredient and/or an active ingredient reservoir; a diffusion barrier that is permeable to said active ingredient and arranged on the skin side of the active ingredient reservoir; and an ergoline derivative or salt thereof as an active ingredient (a) for producing an agent for obtaining and maintaining the circadian rhythm under dopamine therapy and (b) to produce an agent for treating restless legs syndrome as well as Periodic Limb Movement Disorder (PLMD) or nocturnal myoclonus. The invention also relates to the use of a means including a transdermal therapeutic system (TTS) containing a dopamine agonist for treating dopaminergic disease states under a special treatment plan.

The term “TTS” mostly denotes percutaneously acting but also transmucosal systems. A TTS typically has a sheet-like structure and is attached to an area of the skin. A TTS mostly includes a matrix containing an active ingredient (e.g., in the form of a salt) and/or an active ingredient reservoir, and a diffusion barrier that is permeable to the active ingredient on the skin side of the active ingredient reservoir. The system can optionally be attached to the skin by an additional skin-side adhesive that is permeable to the active ingredient. Alternatively, the matrix and/or diffusion barrier can itself have adhesive properties. A non-adhesive TTS can be attached to the skin using other auxiliary means such as adhesive tapes or bandages. The matrix is a material in which the active ingredient is immobilized. An active agent in an active ingredient reservoir however is not necessarily immobilized, which is why the active ingredient reservoir must be enclosed. The diffusion barrier forms the skin-side portion of this shell.

Importantly, all other parts of the shell should be as impermeable as possible, including diffusion paths, to the active ingredient. Immobilized means in this context that any uncontrolled active ingredient flow is prevented. However diffusion of an active agent in a matrix and/or through a diffusion barrier is not only possible but intended. The diffusion coefficients eventually determine the active ingredient flux from the TTS into a patient's skin. The dose released into a patient's skin is in first a approximation a linear function of the active area of the TTS. The active area is the contact area of those TTS portions that allow active ingredient diffusion. TTSs can be used in human and veterinary medicine.

The flux values obtained using a TTS are considerably lower than the values obtain from applying a solution. WO 91/00746 disclosed a device containing lisuride. The flux values for human skin samples specified therein cannot be directly transferred to any achievable in-vivo values.

Diseases for which a dopamine therapy is indicated such as Parkinson's disease are severe chronic and disabling diseases from which older and polymorbid patients suffer frequently. The state-of-the-art practice is oral administration of a combination of dopaminergic substances. These generally include various formulations of levodopa (high initial flux rate, normal or slow release), levodopa boosters such as decarboxylase inhibitors as the base and optionally COMT inhibitors or MAO-B inhibitors, and various dopamine agonists such as bromocriptine, lisuride, cabergoline, pergolide, ropinirole, pramipexole as well as amantadine and, occasionally, anticholinergic agents. The pharmacokinetics of fast-acting levodopa is hard to control for various reasons, and dopamine agonists frequently do not allow safe bioavailability and thus efficacy predictions. All these active agents also can interact for pharmacological and pharmacokinetic reasons, in addition to their interaction with other active agents or pharmaceuticals that older patients with multiple diseases frequently need.

TTSs of the design described-above are used for various indications including Parkinson's disease. When treating Parkinson's disease, the higher doses may be required than the doses required to treat other indications described herein. A transdermal therapeutic system also improves compliance, which is of critical importance for any combinatory treatment of this disease as patients tend to be older and have multiple diseases. Improved control and the chance to reach circadian profiles (e.g. by low stimulation as constantly as possible at night or during a break) are particularly important and have not yet been achieved (e.g. to prevent psychoses and improve the quality of sleep). The ergoline derivatives of the Formula I, such as lisuride, terguride, and bromerguride, have a partially dopamine-agonistic or partially antagonistic effect that contributes to preventing the development of psychoses and can improve existing psychoses and similar problems.

Restless legs syndrome (RLS) is a neurological disease that can occur at all ages but is more frequent in older people; its main symptoms are cramps and pain in the legs due to dysesthesias and paresthesias that trigger an urge to move. As these symptoms mostly occur in periods of reduced activity such as when sitting or resting, the urge to move results in restlessness during the day and sleep disturbances at night. This considerably impairs the quality of life of those, affected. Periodic Limb Movement Disorder (PLMD) or nocturnal myoclonus is associated with RLS and is the ‘kicking’ that occurs during sleep in at least 50% of the people that have RLS.

In addition, peroral dopaminergic therapies often lead to rebound problems on the following day and to so-called augmentations, i.e. hypertonus, restlessness and an urge to move.

It is the technological problem of this invention to provide an agent for the treatment of restless legs syndrome, Periodic Limb Movement Disorder (PLMD), or nocturnal myoclonus, which is free of side effects or at least shows considerably reduced side effects as compared to oral administrations, that has a slow initial flux rate and can be controlled well in terms of quantity administered and effective time.

A transdermal therapeutic system according to the invention described below can ensure an individually desired and controlled effective time (if required, by removing the patch).

Bioavailability is increased by the TTS as compared to peroral administration, which typically reduces the overall dose required to achieve the desired therapeutic and/or prophylactic effect.

A special benefit this invention offers is that—other than with the common one-time oral intake per day—a continuous active ingredient flux is established so that plasma concentrations can be set as defined and variations can be controlled. This mainly prevents the side effects typically observed with one-time oral administration such as fatigue, dizziness, vomiting, constipation, etc. These side effects can be prevented when the level of active ingredient in the plasma is not subject to any major and rapid variation, an automatic occurrence with oral administration, but is set slowly and continuously. In addition, the problems encountered with oral administration such as greatly varying absorption rates and a not too well-defined time of maximum concentration in the plasma depending on the type and time of food intake are virtually eliminated by this invention. Most of all, it prevents overdosing (and thus REM suppression and other disruptions of the sleep pattern). Furthermore, administration can easily be canceled by just removing the TTS. Unlike discontinuing an orally administered active agent, decomposition in the plasma is fast and controlled, which also prevents a hangover, rebound, or augmentation effect.

In the treatment of Parkinson's disease in which dopamine drugs and combinations thereof are taken throughout the day, concentrations in the plasma are not constant but subject to great variation, and this not only for kinetic reasons (highly variable first pass effect depending on the metabolization type) but also depending on individual administration conditions (type and times of food intake, effect of other drugs on resorption and metabolism, etc.). This is why there is a risk of temporary overdosing, which may result in REM suppression and the resulting sleep disturbances or psychoses.

In addition, currently used dopamine therapies frequently have lasting and severe side effects. This is where a transdermal therapeutic system according to the invention described below can ensure individually dosable, adjustable, and controlled action time (if required, by removing the patch) without influencing the circadian rhythm that is often disturbed as a result of treating Parkinson's disease and other dopaminergic diseases. The dopaminergically treatable disease may be a disease from the group consisting of Parkinson's disease, parkinsonism, restless legs syndrome, periodic limb movement disorder or nocturnal myoclonus, and disturbances of the dopaminergic system.

It is the technological problem of the invention to provide an agent for obtaining and maintaining the circadian rhythm that can be individually dosed and adjusted and whose efficacy period can be controlled so that circadian disturbances that occur under dopamine therapy when treating dopaminergic diseases, in particular, when treating patients with Parkinson's disease, are prevented. The α-adrenolytic effect of lisuride and the ergoline derivatives of the Formula I has another benefit for this application in that it also noticeably diminishes urinary urgency at nighttime and other bladder dysfunctions that are rather common in Parkinson patients (such as prostatic hyperplasia), which adds to the success of the therapy.

The technological problem is solved according to the invention in that a transdermal therapeutic system (TTS) is used comprising a pharmaceutical layer containing at least one matrix having an active ingredient, and/or an active ingredient reservoir; a diffusion barrier which is permeable to active ingredients and which is arranged on the skin side of the active ingredient reservoir; and an ergoline derivative according to Formula I or physiologically compatible salt thereof with an acid, 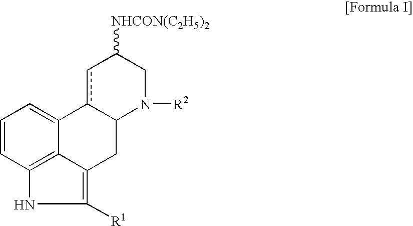
wherein [symbol] is a single or double bond wherein R1 is a H atom or a halogen atom, particularly a bromine atom, and wherein R2 is a C1-C4 alkyl, particularly methyl. Suitable salts of the active ingredients include sulfates, phosphates, maleates, citrates and succinates, especially hydrogen maleate.

The TTS is used as means of obtaining and maintaining the circadian rhythm under continuous dopamine therapy. It is further preferred to select the matrix and/or diffusion barrier so that the transdermal flux F through human skin measured as described in Example 1 is in the range from 0.1 to 5.0 μg/cm²/h, preferably 0.5 to 2.5 μg/cm²/h. A patch with these specifications is particularly suited for obtaining continuous lisuride concentrations in the plasma in the range from 0.05 to 5.0 ng/ml, preferably 0.1 to 0.5 ng/ml. The use of a TTS comprising a matrix and an ergoline derivative of the Formula I or salt thereof as the active ingredient. It is preferred to select the flux F and the active surface area for reaching an effective dose in the range from about 10 μg to about 2 mg of active ingredient (such as lisuride), preferably 50 μg to 1 mg, and most preferably a does less than 1 mg throughout the day or over 24 hours in the patient's system on the second day of application.

The TTS is also used as an agent for treating restless leg syndrome and Periodic Limb Movement Disorder. For such a TSS, it is preferred that the matrix and/or diffusion barrier are selected so that the transdermal flux F through human skin measured as described in Example 1 is in the range from 0.1 to 2.0 μg/cm²/h. It is easy to administer exact individual doses by selecting the flux F and/or the active surface area. It is preferred to select F and active area so that a dose in the range from 10 μg to 1 mg of active ingredient (for example, lisuride), most preferred 50 to 200 μg, is built up per day.

The invention is based on the surprising finding that circadian disturbances under dopamine therapies can be prevented using an ergoline derivative of the Formula I or a salt thereof that is highly effective and has a short half-life (0.5 to 4 hours, particularly 1 to 2 hours). A special benefit this invention offers is the establishment of a continuous active ingredient flux so that plasma concentrations can be set as defined and variations can be controlled. This mainly prevents the dopaminergic side effects such as fatigue, dizziness, etc. that are observed with single oral administrations or using a TTS containing an active ingredient with a long half-life. These side effects can be prevented when the level of active ingredient in the plasma is not subject to any major and rapid variation, an automatic occurrence with oral administration, but is set slowly and continuously. In addition, the problems encountered with oral administration such as greatly varying absorption rates and a not too well-defined time of maximum concentration in the plasma depending on the type and time of food intake are virtually eliminated by this invention. Most of all, it prevents overdosing (and thus REM suppression and other disruptions of the sleep pattern). Furthermore, administration can easily be canceled by just removing the TTS. The drop in agent concentration in the plasma when removing the TTS is further accelerated because of the short half-life of the suitable agents according to the invention. Unlike discontinuing an orally administered active agent or an active agent with a long half-life, decomposition in the plasma is fast and controlled, which also prevents a hangover. Finally it is easy to administer exact individual doses by selecting the flux F and/or the active surface area.

It is preferred to select the flux F and the active surface area for reaching an effective dose in the range from 10 μg to 1 mg of active ingredient (such as lisuride), preferably 50 μg to 1 mg, throughout the day or over 24 hours in the patient's system on the second day of application. In one embodiment of the present invention, it is preferred to select the matrix and/or diffusion barrier so that the transdermal flux F through human skin measured as described in Example 1 is in the range from 0.1 to 5.0 μg/cm²/h, preferably 0.5 to 2.5 μg/cm²/h. A patch with these specifications is particularly suited for obtaining continuous lisuride concentrations in the plasma in the range from 0.05 to 5.0 ng/ml, preferably 0.1 to 0.5 ng/ml. The use of a TTS comprising a matrix and an ergoline derivative of the Formula I or salt thereof as the active ingredient.

Restless legs syndrome has been treated with a single oral administrations of dopaminergic drugs such as lisuride in the evening reduces the symptoms and has a positive influence on the patients quality of life. Unlike the treatment of Parkinson's disease where dopaminergic pharmaceuticals and combinations thereof are administered throughout the day, one-time peroral intake of these drugs for the treatment of restless legs syndrome impairs the building of a tolerance against acute dopaminergic side effects (due to the initial flux rate); this means that the known side effects such as orthostasis, hypotonia, dizziness, nausea, and vomiting may occur with each effective dose. Unpredictable and uncontrollable sleep attacks that have recently been reported more frequently may also occur. Furthermore, agent concentration in the plasma is not constant but subject to great variation, not only for kinetic reasons but also depending on the conditions of drug intake (type and time of food intake, etc.). This is why there is a risk of temporary overdosing, which may result in REM suppression and the resulting problems and sleep disturbances.

The list of ergoline derivatives that can be used includes the following: lisuride, bromolisuride (3-(2-bromo-9,10-didehydro-6-methyl-8α-erg-olinyl)-1,1-diethyl urea), terguride (3-(6-methyl-8α-ergolinyl)-1,1-diethyl urea) and proterguride (3-(6-propyl-8α-ergolinyl)-1,1-diethy-1 urea). However it is preferred when the ergoline derivative is lisuride (3-(9,10-didehydro-6-methyl-8α-ergolinyl)-1,1-diethyl urea) or a physiologically compatible salt thereof with an acid. The production of lisuride and other suitable ergolines according to the invention is described, inter alia, in U.S. Pat. No. 3,953,454, EP 056 358 and U.S. Pat. No. 4,379,790. Suitable salts of the ergoline derivative include sulfates, phosphates, maleates, citrates and succinates, especially hydrogen maleate.

The TTS can be designed as follows. A covering layer can be arranged on the side of the matrix and/or active ingredient reservoir facing away from the skin. The covering layer may be formed by films of polyethylene or polyester. It is typically 10 to 100 microns in thickness. The covering layer may be pigmented and/or metal plated to ensure sufficient protection from light. Metal plating involves applying a very thin layer (typically less than 1 micron, mostly in the 10-100 nm range) of a metal such as aluminum to the covering layer. Pigments can be all pigments commonly used for coating including effect pigments as long as these are physiologically harmless. A detachable liner such as a siliconized or fluoropolymer-coated protective film can be provided on the application side.

The matrix and/or diffusion barrier may comprise as their main matrix component a substance selected from the group consisting of polyacrylate, polyurethane, cellulose ether, silicone, polyvinyl compounds, silicate and mixtures of these substances as well as copolymers of these polymeric compounds, preferably polyacrylate; preferably hydrophilic polyacrylate with basic substituents. A main matrix component makes up at least 50 percent by weight, e.g. at least 80-90 percent by weight of the matrix,. The desired flux is set by selecting the substance depending on the diffusion coefficient of the active ingredient and, if required, by selecting the layer thickness of the matrix in orthogonal direction to the skin surface. Matrix thickness is typically in the range from 10 to 500 microns.

A preferred polyacrylate adhesive as main matrix component is commercially available under the brand name GELVA® multipolymer solution 7881, provided by Monsanto Deutschland GmbH, Dusseldorf. We expressly refer to the product sold under this name and its datasheet in the version of Apr. 23, 1996. Eudragit® E100, provided by Rohm, Germany, is a copolymerisate from dimethyl aminomethyl methacrylate with neutral methacrylate esters and particularly well suited for use.

The polyacrylate adhesives listed above provide an advantageous non-trivial combination of properties, namely optimum flux, good adhesive power, good skin compatibility, and durability.

The diffusion barrier can alternatively comprise as its main barrier component a polymer selected from the group consisting of cellulose ester, cellulose ether, silicone, polyolefin and mixtures as well as copolymers of these substances. What has been said about the term of the main matrix component above analogously applies to the term of the main barrier component. The diffusion barrier can be a film with a thickness from 10 to 300 microns; the actual film thickness is selected (in conjunction with the diffusion coefficient of the active ingredient in the polymer) according to the desired flux.

The matrix and/or active ingredient reservoir and/or diffusion barrier can contain the-common adjuvants used in TTSs. It is preferred to use a penetration-enhancing agent that is preferably selected from the group consisting of C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6 alkyl monools, dicarboxylic acid dieesters from C4-8 dicarboxylic acids and C1-6 alkyl monools, and mixtures of these substances. Penetration-enhancing agents improve the flux of the active-ingredient through the skin to which the TTS is attached. Examples of the substances listed above are: 1,2-propane diol, menthol, dexpanthenol, benzyl alcohol, lauryl alcohol, isocetyl alcohol, cetyl alcohol, mineral oil, lauric acid, isopalmitic acid, isostearic acid, oleic acid; methyl ester, ethyl ester, 2-hydroxyethyl ester, glycerol ester, propyl ester, isopropyl ester, butyl ester, sec. butyl ester or isobutyl ester of lauric acid, myristic acid, stearic acid, or palmitic acid. Use of dimethyl isosorbide, isopropyl myristate and lauryl alcohol is preferred, use of lauryl alcohol is most preferred. Other adjuvants are, for example, crystallization inhibitors. Suitable crystallization inhibitors are highly dispersed silicon dioxide or macromolecular substances such as polyvinyl pyrrolidone, polyvinyl alcohols, dextrines, dextranes, sterines, bile acids and, in particular, vinyl pyrrolidone vinylacetate copolymers and polyvinyl pyrrolidone vinylacetate copolymers such as Kollidon® VA 64.

Suitable crystallization inhibitors are highly dispersed silicon dioxide or macromolecular substances such as polyvinyl pyrrolidone, polyvinyl alcohols, dextrines, dextranes, sterines, bile acids and, in particular, polyvinyl pyrrolidone vinylacetate copolymers such as Kollidon® VA 64.

It goes without saying that the penetration-enhancing agent has to be able to diffuse to a sufficient extent through the matrix or diffusion barrier. If a matrix and lauryl alcohol as an adjuvant are used, it is preferred that the lauryl alcohol makes up 10 to 30 percent by weight, most preferred 15 to 20 percent by weight, of the matrix.

In addition to the ingredients listed above, sufficient quantities of sulfur-containing amino acids such as cysteine, methyl donors such as methionine, or antioxidants such as glutathione or sodium hydrogensulfite are added to the matrix as antioxidants because studies have surprisingly shown that this can prevent or dramatically reduce the formation of toxic oxidation products of lisuride such as lisuride-N-oxide. Antioxidants like glutathione can also have a synergistic effect on Parkinson's disease as oxidative stress plays an important part here; even from early stages on, there is a glutathione shortage in the dopaminergic substantia nigra. Methionine again is particularly desirable as a methyl donor because levodopa is mainly decomposed through oxygen methylation (COMT); homoserine levels increase due to the required levodopa quantities (daily dose up to the gram range), which is suspected to be a risk factor for cardial and cerebral events.

The adjuvants can basically make up from 0 to 50 percent by weight of the matrix. The active ingredient can make up 0.2 to 20 percent by weight, preferably 1 to 10 percent by weight, of the matrix. The sum total of main matrix component, adjuvants and active ingredients is always 100 percent by weight.

The active ingredient dose in a human body carrying a TTS is dependent on the diffusion-related properties of the TTS mentioned above and also on its active surface area on, the skin. Active surface area means the area over which the matrix or diffusion barrier comes to rest on the skin. Variation in accordance with the desired dosage will preferably be in a range from about 1 to about 100 cm^{2.}

Within the scope of this invention, a physician can easily set up personalized dose variations for a flux adjusted to the given indication by selecting a suitable patch size. Thus the treatment can easily be adjusted to different body weights, age groups, etc. It is particularly feasible to equip a TTS comprising a (rather large) standard area with subdivision markers for partial doses so that a user can just remove the protective film from a partial area corresponding to the specified dose. The respective subsections can easily be printed on the covering layer.

The use of lisuride, its salts or derivatives with comparably favorable properties as active ingredients offers the following therapeutic benefits. These substances can be applied at extremely low doses (for lisuride: from 0.075 mg orally at a high first pass effect) due to their extraordinarily strong affinity for dopamine and other monoamine receptors; thus a TTS with a relatively small application area can easily build an effective and well adjustable active ingredient level across the area over 24 hours or longer. Unlike long-acting oral active ingredients such as cabergoline, transdermal dosing of lisuride not only is much improved (elimination of the considerable and highly variable first pass effect after oral administration of cabergoline or the like), the effects can also easily be discontinued whenever required (e.g. when side effects occur) by removing the patch. Then the short half-life of lisuride in the blood (ca. 2 hrs) comes in useful—a great contrast to oral dopamine agonists where side effects last for days once they are administered.

The combination of these effects has surprisingly resulted in combining the benefits of continuous and long-lasting dopaminergic stimulation with the other benefits of short-term acting dopaminergic pharmaceuticals in one application. Combining these properties enables physicians to tailor the application to a patient's individual situation and needs as they can select the application scheme of two patches (simultaneous removal and reattachment, overlapping replacement or replacement at an interval) or, even better, to obtain almost any circadian rhythm of dopaminergic therapy by modifying the initial flux rate of the TTS formulation:

A Continuous stimulation when the initial flux rate of the patch matches the terminal half-life after patch removal (tmax-t/2, optionally a short interval, or when simultaneously applying a new TTS with a relatively high initial flux rate);

B A phase with enhanced stimulation (e. g. when adjusting the therapy or for bridging a patient's ‘off’ phase) by applying the second patch while the first is still attached to the skin or by using patches with a high initial flux rate (tmax<<t/2) or very low initial elimination rate (e. g. when the application area is small and the diffusion of the active ingredient increases with the decrease of the concentration gradient); and

C A phase of reduced dopaminergic stimulation such as reducing time-of-day-specific side effects by either complying with an interval between patch removal and attachment of the new patch, or, even simpler, by simultaneously using the new patch with a very low initial flux rate (tmax>>t/2) at the time of removal.

Another advantage of one embodiment of the present invention is the use of one active ingredient with suitable receptor affinity, efficacy and kinetics and opening all options of an easily applicable and well adjustable dopamine treatment for the patient. As the side effects that are almost inevitable when using state-of-the-art oral and transdermal therapies are prevented, stronger efficacy and a clearly improved therapeutic effect are obtained with simple means.

This means that levodopa therapy and its long-term complications can be prevented or delayed or that this or any other oral dopamine therapy has to be applied at low doses only and is thus more compatible.

In this context, the invention also includes a TTS set for obtaining and maintaining a continuous receptor stimulation with circadian rhythm, particularly for Parkinson's disease, said set containing multiple TTS elements that are set up for releasing different doses. The TTS elements can be separated for this purpose, each TTS element being configured for a continuously ascending sequence of F ranging from 0.1 to 5 μg/cm²/h. In addition, or separately, TTS elements can be equipped with a continuous sequence of differing active areas. In the latter case it is possible to use uniform F values. The TTS elements can be arranged on a big TTS design showing markings that indicate the areas to be used. An embodiment in which these elements are separated is conceivable as well, of course.

The invention can also be used for other indications. One application is the use of a TTS according to the invention to produce an agent for the treatment or prevention of the premenstrual syndrome or its symptoms, wherein F preferably is in the range from 0.1 to 0.5 μg/cm²/h, another one to produce an agent that inhibits lactation, wherein F preferably is in the range from 0.1 to 0.5 μg/cm²/h.

Another embodiment of the present invention provides a combination of a transdermal therapeutic system and an oral and/or parenteral preparation containing dopamine agonists for the treatment of dopaminergic disease states

Either a continuous or a discontinuous stimulation may be required depending on the stage of the disease and the actual status of the patient. A good foundation is laid when the level of dopaminergic agents is kept stable across the entire day. However patients frequently report that they often need to take a fast-acting dopaminergic agent at certain times of the day to overcome acute motoric disturbances, severe and painful dystonia, etc. (“kick”). In extreme cases, such sudden “off” states of motoric performance and akinesia (sometimes predictable early in the morning or afternoon, but frequently all of the sudden and unexpectedly) can only be controlled with injectable active agents such as apomorphine. On the other hand, strong and fast efficacy hikes can cause disturbing side effects (e.g. nausea, emesis, orthostatic hypotension, narcoleptic attacks). Overdoses due to the narrow therapeutic time window of all these dopaminergic agents can result in severe dyskinesia, dystonia or, especially in older patients, psychoses. The latter severe problem is mainly connected with high active agent concentrations in the plasma over night that can destroy regular sleeping patterns and prevent the REM sleep phase (with REM rebound during daytime as indication of a psychosis).

Because of the interrelations described, a practical dopamine treatment is started at very low doses of one or several active agents with subsequent, for example, weekly, dose increases until side effects indicate bioavailability. After a subsequent and rather arbitrary reduction of the dose or dose stabilization, the next active agent is administered and set or dosed (“titrated”) accordingly. As a result, treatment plans and most of all dosages vary considerably depending on the severity of the disease, the patient's individual body constitution and metabolization type. Mostly 3 or more different active agents are administered orally. A typical patient would for example start with fast-acting levodopa in the morning, followed by a dose of MAO-B inhibitor and, throughout the day, four or five doses of normally acting levodopa in combination with a dopamine agonist and, eventually, a slow-acting preparation containing levodopa (or a low dose of a long-term acting dopamine agonist) at bedtime ensuring sufficient mobility in the sleep and consequently a high relaxation value.

Such a complicated treatment plan is more often the rule than an exception and is not very well compatible, especially not with older patients, is unstable and sensitive to interaction with other factors such as other agents administered or infection-related diseases as well as dehydration by inadequate fluid intake or excessive fluid loss or liver or kidney dysfunctions. This is unsatisfactory for obvious reasons for both the physicians and the patients. Patients must therefore often be adapted to side effects over several weeks as indoor patients in more or less specialized hospitals.

It is the technological problem of the invention to provide an agent and a treatment plan for treating dopaminergic disease states while preventing or at least reducing disturbing side effects, controlling the initial flux rate of the active agent and keeping good control of agent levels in the plasma and effective time.

The invention solves this technological problem by using a dopamine agonist in the form of an agent, comprising at least two discrete compositions, of which one is a transdermal therapeutic system (TTS) containing the dopaminergic agent and another one containing the same dopaminergic agent and suitable for oral and/or parenteral administration, both suitable for the treatment of dopaminergically treatable diseases with the following elements: a) the TTS is continuously applied, b) within the duration of application in a) the composition for oral or parenteral dosage is administered. Phase b) preferably begins 7 days, more preferably 14 days, most preferably 28 days after the start of phase a). The invention involves in this context the use of a dopamine agonist in the form of an agent consisting of at least one spatially discrete composition, of which one is a transdermal therapeutic system (TTS) containing the dopaminergic agent for the treatment of dopaminergically treatable diseases with the following elements: a) the TTS is continuously applied, b) within the duration of application in a), no dopaminergic agent is applied that differs from the dopamine agonistic agent of the TTS.

Continuous application means that a new TTS is applied before the agent level in the plasma drops disturbingly due to the consumption of the previous TTS, such as below the 0.25-fold of the maximum plasma concentration.

The invention is based on the surprising finding that dopaminergically treatable diseases, particularly Parkinson's disease, can be treated better using a single dopaminergic agent that is highly effective and has a short half-life in the plasma, if the combination of the invention is optionally carried out using one of the treatment plans according to the invention. This means it is important that no other agent than the active ingredient of the TTS is used for treating dopaminergic dysfunctions during the treatment period. Lasting or continuous dopaminergic stimulation is achieved using the TTS. It provides agent concentrations in the plasma that can be well controlled or adjusted. The concentration in the plasma can easily be dosed by varying, for example, the effective surface area of the TTS or its size.

A transdermal and an oral or parenteral form of application of an active ingredient can easily be offered as one kit for a monotherapy of dopaminergic diseases.

The invention also relates to a combination of a transdermal therapeutic system and an oral and/or parenteral preparation containing one and the same dopamine agonist with a short half-life to produce a pharmaceutical for the treatment of dopaminergic diseases.

Furthermore, a slow increase of the concentration of the active agent in the plasma (over days and weeks) can be achieved by applying the TTS; the benefit is that initial side effects are prevented. Moreover, daily application at relatively early times (e.g. between 6.00 a.m. and 3.00 p.m.), for example, can reliably prevent undesirable overstimulation at night and the risk of psychotic states.

The treatment is supplemented as may be required in advanced stages of a disease by administering oral or parenteral preparations with the same dopaminergic agent. The tablets comprise a preferred tmax of 15 to 120 minutes, particularly preferred of 30 to 60 minutes, and a preferred half-life of 0.5 to 4 hours, particularly preferred 1 to 2 hours. tmax indicates the period of time between oral administration and the buildup of the concentration of the tablet's active agent in the plasma. Half-life is the period of time during which the concentration in the plasma drops by half in the descending portion of the time function. Motoric blockages and akinesia are removed whenever required by such oral administration and the fast extra action as needed.

If oral administrations is started only after starting the continuous application of the TTS, considerable tolerance against dopaminergic side effects has built up and it is no longer required to carry out tedious titrations (sometimes over several months) as would be required for setting up different dopaminergic agents under a combinatory therapy. This makes the treatment particularly well tolerable.

Where indicated—for example, because of the severity of an acute condition (e.g. akinesia or dystonia in the morning or during off periods at other times), the same active agent may be administered parenterally (i.m., i.v., subcutaneously, as contained in the TTS). The same benefits apply in principle as described for oral administration. tmax is typically less than 15 minutes, mostly less than 5 minutes.

Lasting side effects, if unexpected side effects occur, can reliably be prevented due to the short half-life of the active agent. A short-term drop of the agent concentration in the plasma is achieved by just removing the TTS. This is a particular advantage over orally administered, long-term acting agents such as pergolide or cabergoline the side effects of which after an administration or overdosage may last several days.

The invention facilitates relatively high total absorption quantities of the active agent as compared to combinatory therapy where it is highly underdosed to prevent side effects resulting from the complex kinetics and interaction of combining different substances. Thus the invention considerably increases clinical efficacy. This fact combined with better tolerability also allows considerably longer treatment with the respective active agent and avoids the use of levodopa formulations. This is particularly important for younger patients with a high remaining life expectancy as levodopa, the gold standard of dopamine therapy) is known to cause long-term effects resulting in severe and unpredictable dyskinesia and hyperkinesia, which makes the patients eventually dependable on outside help and confines them to bed. Animal experiments have also shown that even short-term levodopa treatment, even at low doses as are common in combinatory therapies, causes lasting priming or sensitization by some kind of inciting mechanism resulting in long-term complications in the motoric and mental dopamine systems. Things being what they are, most patients have to rely on levodopa administrations within the first years of the disease and are exposed to the detrimental long-term disadvantages of levodopa due to the underdosage of active agents administered to avoid side effects.

Despite the relatively high total absorption quantities compared to the underdosage practice that is the state of the art, the actual dosage load can be kept low (about≦10 mg per day, particularly preferred is about≦5 mg per day) so that the treatment is relatively independent of any liver or kidney dysfunctions. Potential interaction with other drugs is rather low and predictable as only one active agent is involved in the treatment according to the invention; interaction with the common other Parkinson agents is completely eliminated.

The matrix and/or diffusion barrier may be selected so that the transdermal flux F through human skin measured as described in Example 1 is in the range from 0.1 to 5.0 μg/cm²/h, preferably 0.1 to 4.0 μg/cm²/h.

It is preferred to arrange a TTS set as part of a means wherein the set contains a multitude of TTS elements and wherein said elements are configured for releasing different doses. The TTS elements can be separated, each TTS element being configured for a continuously ascending sequence of F ranging from 0.1 to 5 μg/cm²/h. It is also conceivable to arrange several TTSs with the same F value in a subgroup wherein the F values of the various subgroups form a continuously ascending sequence and other subgroups comprise constant F values, their value being the maximum of the sequence mentioned above. It is preferred to select F and the active area of the TTS so that a dose in the range from 10 μg to 2 mg of active ingredient (such as lisuride) builds up during the day or within 24 hours as from the second day of application, and that this dose subsequently rises in steps. The TTS elements can also have a continuous sequence of different active areas. These may also be divided into subgroups as described above. Suitable according to the invention are also other transdermal forms of application known from the state of the art.

The preparation for oral administration can either be in the form of a tablet, a powder, a capsule or a solution, is formulated using known state-of-the-art methods as required for the respective form of application, and as a tablet preferably contains 25 to 1000 μg of the dopaminergic agent (per tablet), resulting in a dose of 0.075 mg to 5.0 mg per day for lisuride, for example.

The preparation for parenteral administration in the form of an injection or infusion solution is formulated in accordance with known methods and preferably contains 25 to 2000 μg of the dopaminergic agent (per ml of solution). For example, the parenteral dose needed to achieve a fast additional effect for lisuride is up to 5.0 mg with a continuous infusion over 24 or 16 hours and from 25 up to 200 μg in a bolus injection for a single application.

It is preferred when the dopamine agonist with a short half-life is an ergoline derivative of the Formula I or a physiologically tolerable salt thereof with an acid, 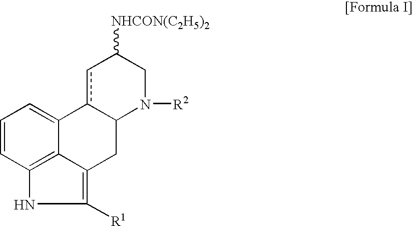
wherein is a single or double bond wherein R1 is a H atom or a halogen atom, particularly a bromine atom, and wherein R2 is C1-4 alkyl, particularly methyl.

The TTS can be applied at various intervals depending on the kinetics of active agent release. It is important that the active agent concentration in the plasma does not show any disturbing variation when the TTS is used continuously. It is preferred that the TTS is applied daily.

The preparation prepared for oral or parenteral administration is preferably administered directly in the event of a dopamine-related malfunction. It may be administered preventively if malfunctions are predictable.

WO 92/20339 discloses a device containing lisuride as the active ingredient and its use for treating Parkinson's disease. Propylene glycol lauric acid increases flux. WO 91/00746 also discloses a TTS containing lisuride. The ergoline compound itself can be formulated in accordance with pharmaceutical methods known as the state of the art.

It is preferred for the TTS to comprise a pharmaceutical layer containing at least one matrix containing the active ingredient and/or an active ingredient reservoir, and a diffusion barrier that is permeable to the active ingredient on the skin side of the active ingredient reservoir; and an ergoline derivative of the Formula I or a salt thereof as an active ingredient.

The TTS can be designed as follows. A covering layer can be arranged on the side of the matrix and/or active ingredient reservoir facing away from the skin. It may be formed by films of polyethylene or polyester. It is typically 10 to 100 microns in thickness. The covering layer may be pigmented, varnished, and/or metal plated to ensure sufficient protection from light. Metal plating involves applying a very thin layer (typically less than 1 micron, mostly in the 10-100 nm range) of a metal such as aluminum to the covering layer. Pigments can be all pigments commonly used for coating including effect pigments as long as these are physiologically harmless. A detachable liner such as a siliconized or fluoropolymer-coated protective film can be provided on the application side.

The matrix and/or diffusion barrier may comprise as their main matrix component a substance selected from the group consisting of polyacrylate, polyurethane, cellulose ether, silicone, polyvinyl compounds, polyisobutylene compounds, silicate and mixtures of these substances as well as copolymers of these polymeric compounds, preferably polyacrylate. A main matrix component makes up at least 50 percent by weight, e.g. at least 80-90 percent by weight of the matrix (matrix to be understood as the finished layer, i.e. main matrix component(s) with adjuvant(s) and active ingredient(s)). The desired flux is set by selecting the substance depending on the diffusion coefficient of the active ingredient and, if required, by selecting the layer thickness of the matrix in orthogonal direction to the skin surface. Matrix thickness is typically in the range from 10 to 500 microns.

A preferred polyacrylate adhesive as main matrix component is one that is commercially available.

The adjuvants can basically make up from 0 to 50 percent by weight of the matrix. The active ingredient can make up 0.5 to 20 percent by weight, preferably 1 to 10 percent by weight, of the matrix. The sum total of main matrix component, adjuvants and active ingredients is always 100 percent by weight.

The invention will be explained in more detail below based on various examples.

### EXAMPLE 1

### Fux Measurement

FRANZ flow-through diffusion cell is used for flux measurement. The measured area is 2 cm² 4 cm² of ventral and dorsal skin of a male hairless mouse (MF1 hr/hr Ola/Hsd, provided by Harlan Olac, UK) are used as our skin sample after carefully removing any subcutaneous fatty tissue. A 2 cm² TTS is applied to the skin sample. The acceptor medium is placed on the opposite side. It is diluted HHBSS (Hepes Hanks Balanced Salt Solution) containing 5.96 g/l of Hepes, 0.35 g/l of NaHCO₃ and 0.1 ml/L10X of HBSS (provided by Gibco, Eggenstein, Del.). Furthermore, 1000 I.U./ml of penicillin (benzylpenicillin potassium salt, provided by Fluka, Neu-Ulm, Del.) are used.

The flux is measured as described below. First, the TTS to be measured is applied to the skin. The skin is mounted in the diffusion cell immediately thereafter. Samples of the acceptor medium are taken at 2-hour intervals between t=0 hrs and t=6 hrs and at 8-hour intervals between t=6 hrs and t=54 hrs. 1 ml of acceptor medium per hour is pumped through the diffusion cell using a peristaltic pump. The temperature of the acceptor medium is controlled using a circulating water bath which keeps the skin at a temperature of 31° C. with an accuracy of 1° C.

The active ingredient concentration in the acceptor medium is determined in accordance with the following specifications using a radioimmunoassay.

Calibration Curves: These are constructed using two different methanol solutions of non-radioactive lisuride hydrogen maleate salt, each containing 1 mg/ml. These solutions are individually diluted with BSA buffer (0.041 M of Na₂HPO₂.2H₂O, 0.026 M of KH₂PO₄, 0.154 M of NaCl, 0.015 M of NaN₃, 0.1% (w/v) of BSA, pH 7, supplemented with 0.05% (w/v) of ascorbic acid) to obtain lisuride-free base concentrations in the range from 1000-3.9 pg/0.1 ml. In addition, a sample without active ingredient (0 pg) is used. The calibration samples are analyzed three times. The lisuride concentrations are calculated using the pharmacokinetic PC program RIO 2.5 (other common software may also be used).

Sample Preparation: The acceptor medium is diluted with BSA buffer prior to the analysis to set the concentrations to an analyzable range of the calibration curve. 100 μl of diluted sample are directly subjected to radioimmunological analysis.

Antiserum: The antiserum (rabbit) is obtained by immunizing with lisuride-1-succinyl-BSA, an immunogen. The antiserum in the assay is diluted 1:12500.

Tracer: ³H-lisuride hydrogen maleate with a specific activity of 4.3 GBq/mg is used.

Incubation: 0.1 ml of BSA buffer with active ingredient, 0.1 ml of tracer solution (ca. 5000 cpm/0.1 ml of BSA buffer) and 0.1 ml of diluted antiserum (1:12500) are added to 0.7 ml of BSA buffer and incubated for 18 hours at 4° C.

Separation: Antibody-bound lisuride is separated from free lisuride by adding 0.2 ml of charcoal suspension (1.25% (w/v) and 0.125% (w/v) of dextrane in BSA buffer) and incubation for 30 minutes at 0° C. The charcoal is sedimented by centrifuging for 15 minutes at 3000 g. The supernatant liquid (containing antibody-bound active ingredient) is decanted and subjected to radiometric analysis.

Radiometric Analysis: 4 ml of Atomlight (NEN) scintillation cocktail are added to the supernatant. The count is carried out using a WALLAC 1409 or 1410 β-scintillation counter without quench control.

Analysis: The percutaneous skin flux is calculated as follows:
F=(C·R)/(A·T),
where F is the percutaneous flux [ng/cm²/h], C the active ingredient concentration in the acceptor medium [ng/ml], R the acceptor medium flow [1 ml/h], A the measured area [2 cm²] and T the sample-taking interval [h].

The maximum transdermal active ingredient flux is obtained directly from the data. Mean percutaneous flux values are determined during days 1 and 2 of the experiment based on the cumulative absorbed dose in time intervals t=0-22 and t=22-54.

Specifications for the Production of TTS

### EXAMPLE 2

### Transdermal Therapeutic System/Device “A”

15 mg of Kollidon VA 64 (crystallization inhibitor) are dissolved in 15 mg of isopropanol. Then 5 mg of lisuride are sprinkled in. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C. for 20 minutes, and finally a covering layer is laminated onto it. Flux measurements as described in Example 1 showed an F value of 0.43 on day 1, 0.44 on day 2, and a maximum F value of 0.85 (each in μg/cm²/h).

### EXAMPLE 3

### Transdermal Therapeutic System/Device “B”

12.5 mg of dimethyl isosorbide are suspended with 2 mg of lisuride in 15 mg of isopropanol. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C. for 20 minutes, and finally a covering layer is laminated onto it. Flux measurements as described in Example 1 showed an F value of 0.23 on day 1, 0.28 on day 2, and a maximum F value of 0.50 (each in μg/cm²/h)

### EXAMPLE 4

### Transdermal Therapeutic System/Device “C”

27.2 mg of polyvinyl pyrrolidone (crystallization inhibitor), preferably Kollidon VA64, and 16.3 mg of lauryl alcohol are dissolved at 60° C. Then 2 mg of lisuride (with or without 0.5 mg of glutathione) is/are dissolved in this solution at 60° C. 39.38 mg of Eudragit E100, 13.41 mg of Citroflex 4A and 1.71 mg of succinic acid are molten at 150-200° C. The lisuride solution is added after the batch has cooled down to 80° C. The product is spread at 80° C. on a siliconized liner using a 500 micron blade. Then the product is cooled down to 20° C.; optionally, a covering layer may be laminated onto it.

Flux measurements as described in Example 1 showed an F value of 0.90 on day 1, 1.6 and 1.76 on day 2, and a maximum F value of 2.4 and 2.53 (each in μg/cm²/h).

### EXAMPLE 5

### Making a Preparation for Oral Administration

A tablet base composition containing lactose, microcrystalline cellulose, corn starch, crosscarmellose and magnesium stearate in the usual quantitative composition is intermixed with 2000 μg of lisuride per each gram of tablet basis composition and pressed into tablets, each of which containing 200 μg of lisuride.

### EXAMPLE 6

### Making a Preparation for Parenteral Administration

An injection base solution containing lactose, NaCl and aqua p.i. in the usual quantitative composition is intermixed with 50 μg of lisuride per gram of injection base solution and filled into amber glass ampoules containing 50 μg of lisuride per ml of solution and preferably lyophilized.

### EXAMPLE 7

### Manufacturing of an Agent According to the Invention

A number of TTSs divided into the four groups as described in Example 2 is put together. The fluxes F of lisuride through human skin of the TTSs of each group comprise are 0.25 μg/cm²/h, 0.5 μg/cm²/h, 0.75 μg/cm²/h and 1.0 μg/cm²/h. At least 7 TTSs are to be in the three groups where F is low. 28 or more TTSs are to be in the group with the highest F. A multitude of tablets from Example 5 and/or a multitude of ampoules from Example 6 is packed with the TTSs compiled in this way. The compilation is accompanied by an instruction sheet that refers to the treatment plan according to the invention.

### EXAMPLE 8

### Treatment of a Patient with Parkinson's Disease

One TTS from Example 7 per day is applied to a Parkinson's disease patient over a period of 28 days. The area of the TTS remains unchanged for seven consecutive days. The TTSs applied in series of 7 consecutive days increase in area so that there will be a four-step increase in lisuride concentration in the plasma (averaged over a day). The lisuride flux F of the TTSs applied in four steps is 0.25 μg/cm²/h, 0.5 μg/cm²/h, 0.75 μg/cm²/h, and 1.0 μg/cm²/h. The daily application of a TTS with an F of 1.0 μg/cm²/h after day 28, i.e. there is no further increase of the dose. Whenever a new TTS is applied, the old one is removed, of course.

After day 28, whenever acute conditions such as severe dystonias occur, a tablet from Example 7 is administered, or the content of an ampoule from Example 7 is injected i.m. Instead, or in addition, a tablet from Example 7 or the content of an ampoule from Example 7 may be administered in the morning for preventive reasons.

The patient will at no time during the treatment show any considerable side effects. The oral or parenteral administrations because of acute conditions are particularly well tolerated, and even after these we did not observe any noticeable disruption of the regular REM sleep.

If against all expectations any disturbing side affects do occur, they can be effectively attenuated by removing the TTS without a replacement, which will soon result in a reduction of agent concentration in the plasma.

What is claimed is:

1. A transdermal delivery device for delivering an effective amount of an ergoline compound to a subject, the device comprising: (a) a backing layer and (b) a matrix adhered to one surface of the backing layer, wherein the matrix comprises: (i) an effective amount of an ergoline compound; (ii) a skin permeation-enhancing amount of a skin penetration enhancer; (iii) a basic polymer selected from the group consisting of a basic polymer a basic copolymer and mixtures thereof and (iv) a lipophilic antioxidant.

2. The transdermal delivery device of claim 1, wherein the device provides substantially continuous transdermal delivery of the ergoline compound to the bloodstream of a mammal, for a predetermined period of time, wherein the effective amount prevents and or alleviates at least one symptom of a disease or condition associated with dopamine deficiency.

3. The transdermal delivery device of claim 1, wherein the matrix further comprises an adhesive.

4. The transdermal delivery device of claim 1, wherein the device further comprises an adhesive layer.

5. The transdermal delivery device of claim 1 wherein the device further comprises a removable protective cover.

6. The transdermal delivery device of claim 1, wherein the device further comprises a diffusion barrier.

7. The transdermal delivery device of claim 1, wherein the basic polymer is selected from the group consisting of a basic polyacrylate, a basic acrylate copolymer.

8. The transdermal delivery device of claim 1, wherein the lipophilic antioxidant inhibits the oxidation of the ergoline compound.

9. The transdermal delivery device of claim 1, wherein the combination of the lipophilic antioxidant and the basic polymer inhibits the oxidation of the ergoline compound.

10. The transdermal delivery device of claim 1, wherein the lipophilic antioxidant is selected from the group consisting of Di-tert-butylmethylphenol (BHT), Di-tert-butylmetoxyphenol, a tocopherol, a ubichinone and mixtures thereof.

11. The transdermal delivery device of claim 1, wherein the lipophilic antioxidant is selected from the group consisting of Vitamin E, α-tocopherol, γ-tocopherol, δ-tocopherol, α-tocopherol acetate, α-tocotrienol, β-tocotrienol, δ tocotrienol, γ-tocotrienol, Nutriene (Tocotrienols), γ-Oryzanol, Trolox, 2,2,5,7,8-pentamethyl-6-chromanol, Di-tert-butylmethylphenol (BHT), Di-tert-butylmetoxyphenol, a ubichinone and mixtures thereof.

12. The transdermal delivery device of claim 1, wherein the lipophilic antioxidant is selected from the group consisting of: 2,2,5,7,8-pentamethyl-6-chromanol; 2,2′-Azobis (2-amidino-propane)dihydrochloride (AAPH); 2,6-di-tert-butyl-4-methylphenol (BHT); ethanolic BHT; 2-tert-butyl-4-methylphenol; 4-Difluoro-5-(4-phenyl-1,3-butadienyl)-4-bora-3a,4a-diaza-s-indacene-3-undecanoic acid; Á-oryzanol; ascorbyl palmitate; α-carotene; β-carotene; Coenzyme Q10; Coenzyme Q10H2; coenzyme Q9 (CoQ9H2); copper; β-Cryptoxanthin; a-lipoic acid; Lutein/zeaxanthin; luteine; lycopene; malondialdehyde (MDA); meth-6-hydroxy-2,5,7,8-tetramethyl-2-carboxylic acid (Trolox); Nutriene (Tocotrienols); T-Oryzanol; Retinol; R-tocopherol acetate; R-tocopherol; selenium; α-tocopherol; a-tocopherol acetate; γ-tocopherol; d-tocopherol; Trolox; ubiquinol; Ubiquinol 10; vitamin E; and zinc.

13. The transdermal delivery device of claim 1, wherein the lipophilic antioxidant is 2,6-di-tert-butyl-4-methylphenol (BHT).

14. The transdermal delivery device of claim 1, wherein the lipophilic antioxidant is 2-tert-butyl-4-methylphenol.

15. The transdermal delivery device of claim 1, wherein the lipophilic antioxidant is present in an amount from about 0.25% to about 5% w/w.

16. The transdermal delivery device of claim 1, wherein the basic polymer is selected from the group consisting of a basic polymers, a basic polyacrylates, a hydrophilic polyacrylate with basic substituents, butyl methacrylate-(2-diamino ethyl)methacrylate-methacrylate-copolymer; a copolymer of dimethyl aminomethyl methacrylate with neutral methacrylate esters; GELVA® multipolymer, and mixtures thereof.

17. The transdermal delivery device of claim 1, wherein the device further comprises an adhesive layer, wherein the adhesive layer comprises a basic polymer.

18. The transdermal delivery device of claim 1, wherein the matrix further comprises a crystallization inhibitor.

19. The transdermal delivery device of claim 1, wherein the matrix further comprises a crystallization inhibitor and the crystallization inhibitor is selected from the group consisting of polyvinyl pyrrolidone, vinyl pyrrolidone vinylacetate copolymers and polyvinyl pyrrolidone vinylacetate copolymers, polyvinyl alcohols, dextrines, dextranes, sterines, and bile acids.

20. The transdermal delivery device of claim 1, wherein the matrix further comprises a crystallization inhibitor and the crystallization inhibitor is Kollidon® VA 64.

21. The transdermal delivery device of claim 1, wherein the matrix further comprises a crystallization inhibitor and wherein the crystallization inhibitor is present in an amount from about 0.25% to about 5% w/w.

22. The transdermal delivery device of claim 1, wherein the matrix further comprises an adhesiveness enhancer.

23. The transdermal delivery device of claim 1, wherein the adhesive layer further comprises an adhesiveness enhancer.

24. The transdermal delivery device of claim 1, wherein the adhesiveness enhancer is selected from the group consisting of resins, polyacrylates and mixtures thereof.

25. The transdermal delivery device of claim 1, wherein the adhesiveness enhancer is present in an amount from about 1% to about 20% w/w.

26. The transdermal delivery device of claim 1, wherein the adhesiveness enhancer is present in an amount from about 2% to about 10% w/w.

27. The transdermal delivery device of claim 1, wherein the ergoline compound is selected from the group consisting of lisuride, proterguride, bromolisuride (3-(2-bromo-9,10-didehydro-6-methyl-8α-erg-olinyl)-1,1-diethyl urea), terguride (3-(6-methyl-8α-ergolinyl)-1,1-diethyl urea) and proterguride (3-(6-propyl-8α-ergolinyl)-1,1-diethy-1 urea).

28. The transdermal delivery device of claim 1, wherein the ergoline compound is selected from the group consisting of lisuride (3-(9,10-didehydro-6-methyl-8α-ergolinyl)-1,1-diethyl urea), a physiologically compatible salt of lisuride, and mixtures thereof.

29. The transdermal delivery device of claim 1, wherein the backing layer is impermeable, further comprising an adhesive layer permeable for the ergoline compound, wherein the ergoline compound is stabilized by the presence of the lipophilic antioxidant and the basic polymer.

30. A method for treating a disease or condition associated with dopamine deficiency, the method comprising the step of adhering the transdermal delivery device of claim 1 onto the skin of a mammal, wherein the device provides substantially continuous transdermal delivery of the ergoline compound to the bloodstream of the mammal, for a predetermined period of time, in a effective amount, wherein the effective amount prevents and or alleviates at least one symptom of a disease or condition associated with dopamine deficiency.

31. A method for treating a dopaminergic disease, the method comprising the step of adhering the transdermal delivery device of claim 1 onto the skin of a mammal, wherein the device alleviates at least one symptom associated with the dopaminergic disease being treated.

32. A method of treating a condition associated with dopamine deficiency, the method comprising the step of adhering the transdermal delivery device of claim 1 onto the skin of a mammal, wherein the device alleviates at least one symptom associated with the dopaminergic disease being treated.

33. A method for treating Parkinson's Disease, the method comprising the step of adhering the transdermal delivery device of claim 1 onto the skin of a mammal, wherein the device alleviates at least one symptom associated with Parkinson's Disease.

34. A method for alleviating and/or preventing the symptoms of Restless Legs Syndrome and/or Periodic Limb Movement Disorder, the method comprising the step of adhering the transdermal delivery device of claim 1 onto the skin of a mammal, wherein the device alleviates and/or prevents at least one symptom associated with Restless Leg Syndrome and/or Periodic Limb Movement Disorder.

35. A method for treating and/or preventing migraines, the method comprising the step of adhering the transdermal delivery device of claim 1 onto the skin of a person who has recurring migraines, wherein the device prevents, aborts and/or alleviates at least one symptom associated with migraines.

36. A method for preventing and/or alleviating the symptoms of Premenstrual Syndrome, the method comprising the step of adhering the transdermal delivery device of claim 1 onto the skin of a female human, wherein the device alleviates and/or prevents at least one symptom associated with Premenstrual Syndrome.

37. A method for inhibiting lactation, the method comprising the step of adhering the transdermal delivery device of claim 1 onto the skin of a female mammal, wherein the device inhibits lactation in the mammal.

38. A method for treating hyperprolactinemia, the method comprising the step of adhering the transdermal delivery device of claim 1 onto the skin of a mammal, wherein the device decreases or inhibits lactation in the mammal.

39. A method for treating gynecomastia, the method comprising the step of adhering the transdermal delivery device of claim 1 onto the skin of a mammal, wherein the device decreases the size of the mammary glands in the mammal.

40. A method for treating a patient with a depression disorder, comprising the step of adhering the transdermal delivery device of claim 1 onto the skin of the patient, wherein the device provides the patient with at least a partial relief from the symptoms of the depression disorder for a predetermined period of time.

41. A method for treating a patient with an attention disorder, comprising the step of adhering the transdermal delivery device of claim 1 onto the skin of the patient, wherein the device provides the patient with at least a partial relief from the symptoms of the attention disorder for a predetermined period of time.

42. A method for treating a person for nicotine dependency during smoking cessation therapy, comprising the step of adhering the transdermal delivery device of claim 1 onto the skin of the patient, wherein the device provides the patient with an effective amount of an ergoline compound.

43. A method for treating a patient with an eating disorder, comprising the step of adhering the transdermal delivery device of claim 1 onto the skin of the patient, wherein the device provides the patient with an effective amount of an ergoline compound.

44. A method for treating a patient with a loss of libido, comprising the step of adhering the transdermal delivery device of claim 1 onto the skin of the patient, wherein the device increases the patient's libido.

45. A method for suppressing bowel movements or diarrhea in a patient with carcinoid syndrome, comprising the step of adhering the transdermal delivery device of claim 1 onto the skin of the patient, wherein the device suppresses the patient's bowel movements or diarrhea.

46. A method for suppressing bowel movements or diarrhea in a patient with irritable bowel syndrome, comprising the step of adhering the transdermal delivery device of claim 1 onto the skin of the patient, wherein the device suppresses the patient's bowel movements or diarrhea.

47. A method for preventing or treating fibrotic cardiac valvulopathy, comprising the step of adhering the transdermal delivery device of claim 1 onto the skin of the carcinoid patient, wherein the device prevents or treats fibrotic cardiac valvulopathy.

48. A method for obtaining or maintaining a patient's circadian rhythm, comprising the step of adhering the transdermal delivery device of claim 1 onto the skin of the patient, wherein the device provides the patient with an effective amount of an ergoline compound.

49. A method for obtaining or maintaining the circadian rhythm of a patient under dopamine therapy, comprising the step of adhering the transdermal delivery device of claim 1 onto the skin of the patient, wherein the device provides the patient with an effective amount of an ergoline compound, wherein the patient is under dopamine therapy.

50. A method for preventing disturbances in the circadian rhythm of a patient under dopamine therapy, comprising the step of adhering the transdermal delivery device of claim 1 onto the skin of the patient, wherein the device provides the patient with an effective amount of an ergoline compound.

51. The transdermal delivery device of claim 1, wherein the lipophilic antioxidant reacts with free radicals.

52. The transdermal delivery device of claim 1, wherein the ergoline compound selected from the group consisting of lisuride, proterguride, physiologically compatible salts thereof, and mixtures thereof.

53. The transdermal delivery device of claim 1, wherein the ergoline compound is according to Formula I or physiologically compatible salt thereof with an acid, 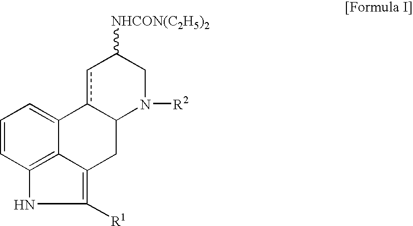
wherein [symbol] is a single or double bond wherein R1 is an H atom or a halogen atom, particularly a bromine atom, and wherein R2 is a C1-4 alkyl, for producing an agent for obtaining and maintaining the circadian rhythm under a continuous dopamine therapy.

54. The transdermal delivery device of claim 1, wherein the matrix is selected to provide a transdermal flux F of the ergoline compound through human skin is in the range from about 0.1 to about 5.0 μg/cm²/h.

55. A transdermal delivery device for delivering an effective amount of an ergoline compound to a subject, the device comprising: (a) a backing layer and (b) a matrix adhered to one surface of the backing layer, wherein the matrix comprises: (i) an effective amount of an ergoline compound; (ii) a skin permeation-enhancing amount of a skin penetration enhancer; (iii) a main matrix component and (iv) a hydrophilic antioxidant.

56. A transdermal delivery device for delivering an effective amount of an ergoline compound to a subject, the device comprising: (a) a backing layer (b) an active ingredient reservoir adhered to one surface of the backing layer, wherein the active ingredient reservoir comprises: (i) an effective amount of an ergoline compound; (ii) a skin permeation-enhancing amount of a skin penetration enhancer; (iii) a main matrix component and (iv) a hydrophilic antioxidant.

57. The transdermal delivery device of claim 55 or 56, wherein the main matrix component is a substance selected from the group consisting of polyacrylate, polyurethane, cellulose ether, silicone, polyvinyl compounds, silicate, mixtures thereof, and copolymers of the polymeric compounds.

58. The transdermal delivery device of claim 55, wherein the main matrix component is a hydrophilic polyacrylate with basic substituents.

59. The transdermal delivery device of claim 55 or 56, wherein the device further comprises a diffusion barrier.

60. The transdermal delivery device of claim 55 or 56, wherein the device further comprises a diffusion barrier, the barrier comprising a synthetic polymer selected from the group consisting of cellulose ester, cellulose ether, silicone, polyolefin, mixtures thereof, and copolymers thereof.

61. The transdermal delivery device of claim 55 or 56 wherein the penetration-enhancing agent is selected from the group consisting of C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6-alkyl monools, dicarboxylic acid diesters from C4-8-dicarboxylic acids, C1-6 alkyl monools, and mixtures thereof.

62. The transdermal delivery device of claim 55, wherein said matrix further comprises a crystallization inhibitor, wherein the crystallization inhibitor is selected from the group consisting of highly dispersed silicone dioxide, polyvinyl pyrrolidone, polyvinyl alcohols, dextrines, dextranes, sterines, bile acids and, in particular, and vinyl pyrrolidone vinylacetate copolymers.

63. The transdermal delivery device of claim 58, wherein the active ingredient reservoir further comprises a crystallization inhibitor, wherein the crystallization inhibitor is selected from the group consisting of highly dispersed silicone dioxide, polyvinyl pyrrolidone, polyvinyl alcohols, dextrines, dextranes, sterines, bile acids and, in particular, and vinyl pyrrolidone vinylacetate copolymers.

64. The transdermal delivery device of claim 55 or 56, wherein the antioxidant is selected from the group consisting of cysteine, methionine, glutathione, sodium hydrogensulfite, sodium sulfite, citric acid, ascorbic acid (vitamin C), alkyl gallate, ascorbyl palmitate, uric acid, TBA-RS, and protein carbonyls.

65. The transdermal delivery device of claim 55, wherein the matrix is selected to provide a transdermal flux F of the ergoline compound through human skin in the range from about 0.1 to about 0.5 μg/cm²/h.

66. A method for preventing and/or alleviating the symptoms of Premenstrual Syndrome, the method comprising the step of adhering the transdermal delivery device of claim 55 onto the skin of a female human, wherein the device alleviates and/or prevents at least one symptom associated with Premenstrual Syndrome.

67. A method for inhibiting lactation, the method comprising the step of adhering the transdermal delivery device of claim 55 onto the skin of a female mammal, wherein the device inhibits lactation in the mammal.

68. A method for treating hyperprolactinemia, the method comprising the step of adhering the transdermal delivery device of claim 55 onto the skin of a mammal, wherein the device decreases or inhibits lactation in the mammal.

69. A method for treating gynecomastia, the method comprising the step of adhering the transdermal delivery device of claim 55 onto the skin of a mammal, wherein the device decreases the size of the mammary glands in the mammal.

70. A method for obtaining or maintaining a patient's circadian rhythm, comprising the step of adhering the transdermal delivery device of claim 55 onto the skin of the patient, wherein the device provides the patient with an effective amount of an ergoline compound.

71. A method for treating circadian disturbances under dopamine therapy, comprising the step of adhering the transdermal delivery device of claim 55 onto the skin of the patient, wherein the device provides the patient with an effective amount of an ergoline compound.

72. The transdermal delivery device of claim 55, wherein the matrix further comprises an adhesive.

73. The transdermal delivery device of claim 55, wherein the device further comprises an adhesive layer.

74. The transdermal delivery device of claim 55 wherein the device further comprises a removable protective cover.

75. The transdermal delivery device of claim 55, wherein the device further comprises a diffusion barrier.

76. The transdermal delivery device of claim 55, wherein the device further comprises an adhesive layer.

77. The transdermal delivery device of claim 55, wherein the matrix further comprises a crystallization inhibitor.

78. The transdermal delivery device of claim 55, wherein the matrix further comprises a crystallization inhibitor and the crystallization inhibitor is selected from the group consisting of polyvinyl pyrrolidone, vinyl pyrrolidone vinylacetate copolymers, polyvinyl pyrrolidone vinylacetate copolymers, polyvinyl alcohols, dextrines, dextranes, sterines, and bile acids.

79. The transdermal delivery device of claim 55, wherein the matrix further comprises a crystallization inhibitor and the crystallization inhibitor is Kollidon® VA 64.

80. The transdermal delivery device of claim 55, wherein the matrix further comprises a crystallization inhibitor and wherein the crystallization inhibitor is present in an amount from about 0.25% to about 5% w/w.

81. The transdermal delivery device of claim 55, wherein the matrix further comprises an adhesiveness enhancer.

82. The transdermal delivery device of claim 55, wherein the adhesive layer further comprises an adhesiveness enhancer.

83. The transdermal delivery device of claim 55, wherein the adhesiveness enhancer is selected from the group consisting of resins, polyacrylates and mixtures thereof.

84. The transdermal delivery device of claim 55, wherein the adhesiveness enhancer is present in an amount from about 1% to about 20% w/w.

85. The transdermal delivery device of claim 55, wherein the adhesiveness enhancer is present in an amount from about 2% to about 10% w/w.

86. The transdermal delivery device of claim 55, wherein the ergoline compound is selected from the group consisting of lisuride, proterguride, bromolisuride (3-(2-bromo-9,10-didehydro-6-methyl-8α-erg-olinyl)-1,1-diethyl urea), terguride (3-(6-methyl-8α-ergolinyl)-1,1-diethyl urea) and proterguride (3-(6-propyl-8α-ergolinyl)-1,1-diethy-1 urea).

87. The transdermal delivery device of claim 55, wherein the ergoline compound is selected from the group consisting of lisuride (3-(9,10-didehydro-6-methyl-8α-ergolinyl)-1,1-diethyl urea), a physiologically compatible salt of lisuride, and mixtures thereof.

88. The transdermal delivery device of claim 55, wherein the backing layer is impermeable, further comprising an adhesive layer permeable for the ergoline compound, wherein the ergoline compound is stabilized by the presence of a hydrophilic antioxidant.

89. A method for treating a disease or condition associated with dopamine deficiency, the method comprising the step of adhering the transdermal delivery device of claim 55 onto the skin of a mammal, wherein the device provides substantially continuous transdermal delivery of the ergoline compound to the bloodstream of the mammal, for a predetermined period of time, in a effective amount, wherein the effective amount prevents and or alleviates at least one symptom of a disease or condition associated with dopamine deficiency.

90. A method for treating a dopaminergic disease, the method comprising the step of adhering the transdermal delivery device of claim 55 onto the skin of a mammal, wherein the device alleviates at least one symptom associated with the dopaminergic disease being treated.

91. A method of treating a condition associated with dopamine deficiency, the method comprising the step of adhering the transdermal delivery device of claim 55 onto the skin of a mammal, wherein the device alleviates at least one symptom associated with the dopaminergic disease being treated.

92. A method for treating Parkinson's Disease, the method comprising the step of adhering the transdermal delivery device of claim 55 onto the skin of a mammal, wherein the device alleviates at least one symptom associated with Parkinson's Disease.

93. A method for alleviating and/or preventing the symptoms of Restless Legs Syndrome and/or Periodic Limb Movement Disorder, the method comprising the step of adhering the transdermal delivery device of claim 55 onto the skin of a mammal, wherein the device alleviates and/or prevents at least one symptom associated with Restless Leg Syndrome and/or Periodic Limb Movement Disorder.

94. A method for treating and/or preventing migraines, the method comprising the step of adhering the transdermal delivery device of claim 55 onto the skin of a person who has recurring migraines, wherein the device prevents, aborts and/or alleviates at least one symptom associated with migraines.

95. A method for preventing and/or alleviating the symptoms of Premenstrual Syndrome, the method comprising the step of adhering the transdermal delivery device of claim 55 onto the skin of a female human, wherein the device alleviates and/or prevents at least one symptom associated with Premenstrual Syndrome.

96. A method for inhibiting lactation, the method comprising the step of adhering the transdermal delivery device of claim 55 onto the skin of a female mammal, wherein the device inhibits lactation in the mammal.

97. A method for treating hyperprolactinemia, the method comprising the step of adhering the transdermal delivery device of claim 55 onto the skin of a mammal, wherein the device decreases or inhibits lactation in the mammal.

98. A method for treating gynecomastia, the method comprising the step of adhering the transdermal delivery device of claim 55 onto the skin of a mammal, wherein the device decreases the size of the mammary glands in the mammal.

99. A method for treating a patient with a depression disorder, comprising the step of adhering the transdermal delivery device of claim 55 onto the skin of the patient, wherein the device provides the patient with at least a partial relief from the symptoms of the depression disorder for a predetermined period of time.

100. A method for treating a patient with an attention disorder, comprising the step of adhering the transdermal delivery device of claim 55 onto the skin of the patient, wherein the device provides the patient with at least a partial relief from the symptoms of the attention disorder for a predetermined period of time.

101. A method for treating a person for nicotine dependency during smoking cessation therapy, comprising the step of adhering the transdermal delivery device of claim 55 onto the skin of the patient, wherein the device provides the patient with an effective amount of an ergoline compound.

102. A method for treating a patient with an eating disorder, comprising the step of adhering the transdermal delivery device of claim 55 onto the skin of the patient, wherein the device provides the patient with an effective amount of an ergoline compound.

103. A method for treating a patient with a loss of libido, comprising the step of adhering the transdermal delivery device of claim 55 onto the skin of the patient, wherein the device increases the patient's libido.

104. A method for suppressing bowel movements or diarrhea in a patient with carcinoid syndrome, comprising the step of adhering the transdermal delivery device of claim 55 onto the skin of the patient, wherein the device suppresses the patient's bowel movements or diarrhea.

105. A method for suppressing bowel movements or diarrhea in a patient with irritable bowel syndrome, comprising the step of adhering the transdermal delivery device of claim 55 onto the skin of the patient, wherein the device suppresses the patient's bowel movements or diarrhea.

106. A method for preventing or treating fibrotic cardiac valvulopathy, comprising the step of adhering the transdermal delivery device of claim 55 onto the skin of the carcinoid patient, wherein the device prevents or treats fibrotic cardiac valvulopathy.

107. A method for obtaining or maintaining a patient's circadian rhythm, comprising the step of adhering the transdermal delivery device of claim 55 onto the skin of the patient, wherein the device provides the patient with an effective amount of an ergoline compound.

108. A method for obtaining or maintaining the circadian rhythm of a patient under dopamine therapy, comprising the step of adhering the transdermal delivery device of claim 55 onto the skin of the patient, wherein the device provides the patient with an effective amount of an ergoline compound, wherein the patient is under dopamine therapy.

109. A method for preventing disturbances in the circadian rhythm of a patient under dopamine therapy, comprising the step of adhering the transdermal delivery device of claim 55 onto the skin of the patient, wherein the device provides the patient with an effective amount of an ergoline compound.

110. The transdermal delivery device of claim 55, wherein the ergoline compound selected from the group consisting of lisuride, proterguride, physiologically compatible salts thereof, and mixtures thereof.

111. The transdermal delivery device of claim 55, wherein the ergoline compound is according to Formula I or physiologically compatible salt thereof with an acid, 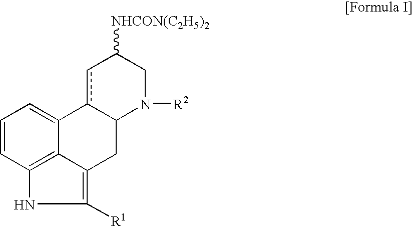
wherein [symbol] is a single or double bond wherein R1 is an H atom,or a halogen atom, particularly a bromine atom, and wherein R2 is a C1-4 alkyl, for producing an agent for obtaining and maintaining the circadian rhythm under a continuous dopamine therapy.

112. The transdermal delivery device of claim 55, wherein the matrix is selected to provide a transdermal flux F of the ergoline compound through human skin is in the range from about 0.1 to about 5.0 μg/cm²/h.

113. A transdermal therapeutic system set for delivering an effective amount of an ergoline compound to a subject, the set comprising a multitude of transdermal therapeutic system elements, wherein said elements are configured for releasing different doses.

114. The transdermal therapeutic system (TTS) set of claim 112, the set further comprising: (a) a backing layer and (b) a matrix adhered to one surface of the backing layer, wherein the matrix comprises separated TTS elements, wherein each, TTS element is configured to provide a continuously ascending sequence of flux F values ranging from about 0.1 ug/cm2/h to about 5.0 ug/cm2/h.

115. The transdermal therapeutic system (TTS) set of claim 112, wherein the elements are separated, each element providing a different flux F value.

116. The transdermal therapeutic system (TTS) set of claim 112 wherein the TTS elements are equipped with different active surfaces in a continuous sequence.

117. A method of treating a patient having a dopaminergically treatable disease, the method comprising administration of a dopamine agonist in at least two spatially discrete compositions, wherein one composition is a transdermal therapeutic system (TTS) containing the dopaminergic agent for the treatment of dopaminergically treatable diseases and at least one compositions is a preparation for oral or parenteral application, wherein said preparation comprises the same dopaminergic agent, and further wherein the preparation for oral or parenteral dosage is administered during the time period in which the TTS is applied to the patient.

118. The method according to claim 1 16 wherein the dopaminergically treatable disease is selected from the group consisting of Parkinson's disease, parkinsonism, restless legs syndrome, and disturbances of the dopaminergic system.

119. The method according to claim 116 wherein the dopamine agonist has a half-life of from about 0.5 hour to about 4 hours.

120. The method according to claim 116 wherein the dopamine agonist has a half-life of from about 1 hour to about 2 hours.

121. The method according to claim 116 wherein a TTS set is provided containing a multitude of TTS elements and wherein these elements are designed for the release of different doses.

122. The method according to claim 116 wherein the preparation in tablet form for oral administration contains 25 to 500 μg of the dopaminergic agent per tablet.

123. The method according to claim 116 wherein the preparation in form of an injection or infusion solution for parenteral administration contains 25 to 2000 μg of the dopaminergic agent per ml of solution.

124. A method of treating a patient having a dopaminergic disease using a combination of compositions, the combination comprising a transdermal therapeutic system and at least one oral or parenteral preparation, wherein both the transdermal therapeutic system and the preparation comprise the same dopamine agonist, wherein the dopamine agonist has a short half-life.
